(19) United States
(12) Patent Application Publication
(10) Pub. No.: US 2025/0229065 A1
(43) Pub. Date: Jul. 17, 2025

(54) DELIVERY DEVICE FOR CONFIRMING TISSUE CAPTURE OF A SHUNT DEVICE

(71) Applicant: EDWARDS LIFESCIENCES CORPORATION, Irvine, CA (US)

(72) Inventors: Morgan Alex Jawitz, Mountain View, CA (US); Daniel Vincent Llanes, Huntington Beach, CA (US); Scott Louis Pool, Laguna Hills, CA (US); Sandra Ahide Alcantar Chavez, Anaheim, CA (US)

(73) Assignee: EDWARDS LIFESCIENCES CORPORATION, Irvine, CA (US)

(21) Appl. No.: 19/096,571

(22) Filed: Mar. 31, 2025

Related U.S. Application Data
(63) Continuation of application No. PCT/US2023/034386, filed on Oct. 3, 2023.
(60) Provisional application No. 63/378,181, filed on Oct. 3, 2022.

(51) Int. Cl. A61M 27/00; A61M 25/00; A61M 25/06

(52) U.S. Cl. CPC A61M 27/002; A61M 25/005; A61M 25/0662; A61M 2202/0007; A61M 2202/04; A61M 2205/0216; A61M 2205/0266; A61M 2205/32; A61M 2205/582

(57) ABSTRACT

A device for delivering a shunt device within a human body, the shunt device having a central flow tube, a proximal arm, and a distal arm, the proximal arm and distal arm configured to capture a tissue wall therebetween. The delivery device includes a catheter that includes a tube and a side opening. The delivery device further includes a flexible wire slidably receivable in the tube and extendable into the side opening. The flexible wire includes a tip at a distal end configured to deflect upon contact with human tissue. The flexible wire is disposed outside of the central flow tube of the shunt device.

### CROSS-REFERENCE TO RELATED APPLICATION(S)

This application is a continuation of International Application No. PCT/US2023/034386, filed Oct. 3, 2023, which claims the benefit of U.S. Provisional Application No. 63/378,181, filed Oct. 3, 2022, the disclosures of which are hereby incorporated by reference in their entireties.

### BACKGROUND

The present disclosure relates generally to implantable devices and more specifically to confirming placement of cardiovascular shunt devices.

Shunt devices can be positioned in the heart to shunt blood between the left atrium and the right atrium to reduce pressure in the left atrium. The left atrium can experience elevated pressure due to abnormal heart conditions caused by age and/or disease. For example, shunt devices can be used to treat patients with heart failure (also known as congestive heart failure). Shunt devices can be positioned in the septal wall between the left atrium and the right atrium to shunt blood from the left atrium into the right atrium, thus reducing the pressure in the left atrium.

### SUMMARY

A device for delivering a shunt device within a human body, the shunt device having a central flow tube, a proximal arm, and a distal arm, the proximal arm and distal arm configured to capture a tissue wall therebetween. The delivery device includes a catheter that includes a tube and a side opening. The delivery device further includes a flexible wire slidably receivable in the tube and extendable into the side opening. The flexible wire includes a tip at a distal end configured to deflect upon contact with human tissue. The flexible wire is disposed outside of the central flow tube of the shunt device.

A method of detecting tissue capture of a shunt device, the shunt device having a central flow tube, a proximal arm, and a distal arm, the proximal arm and distal arm configured to capture a tissue wall therebetween. The method includes advancing a flexible wire through a tube of a catheter and the proximal arm of the shunt device, and imaging, by fluoroscopy, the advancement of the flexible wire.

A device for delivering a shunt device within a human body, the shunt device having a central flow tube, a proximal arm, and a distal arm, the proximal arm and distal arm configured to capture a tissue wall therebetween. The delivery device includes a catheter including a tube extending outward from the catheter, the tube having an open distal end disposed adjacent to and outside of the central flow tube of the shunt device. The delivery device further includes a flexible wire slidably receivable in the tube, the flexible wire including a tip at a distal end configured to deflect upon contact with human tissue.

### BRIEF DESCRIPTION OF THE DRAWINGS

### Anatomy of Heart H and Vasculature V

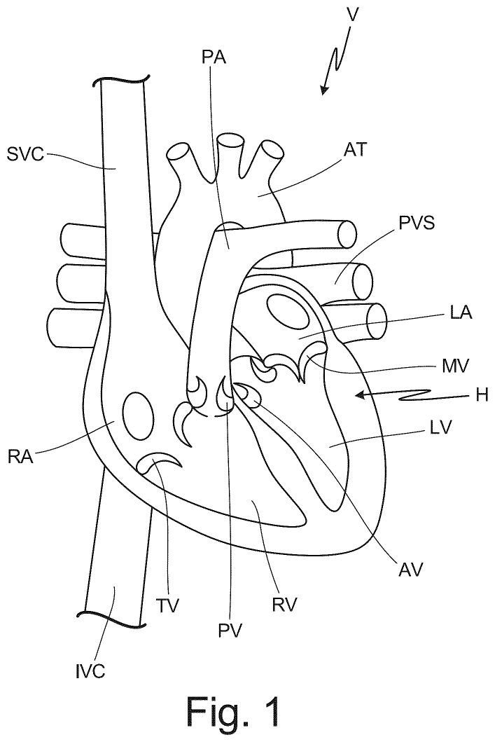

FIG. 1 is a schematic diagram of a heart and vasculature.

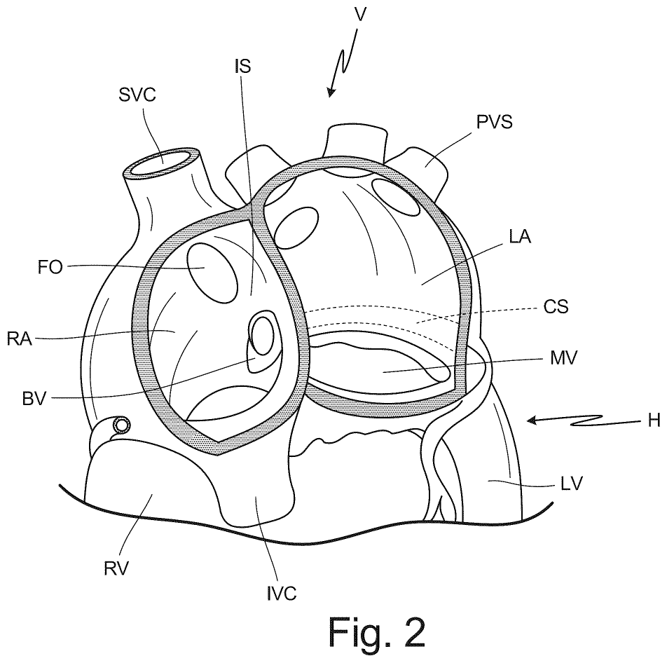

FIG. 2 is a schematic cross-sectional view of the heart.

### Shunt Devices 100 and 100′

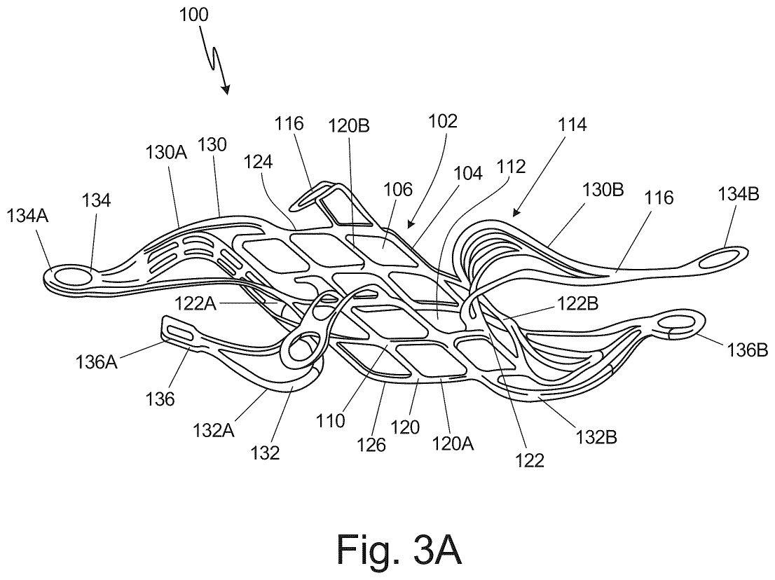

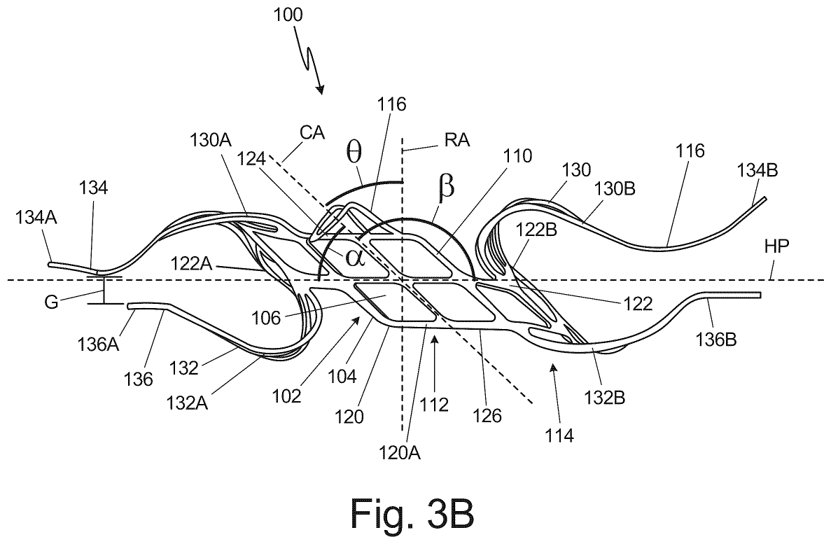

FIG. 3A is a perspective view of a shunt device.

FIG. 3B is a side view of the shunt device.

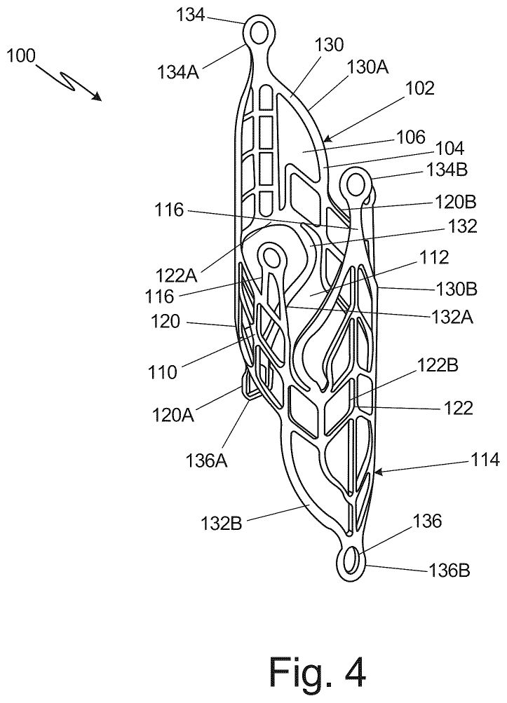

FIG. 4 is a perspective view of the shunt device in a collapsed configuration.

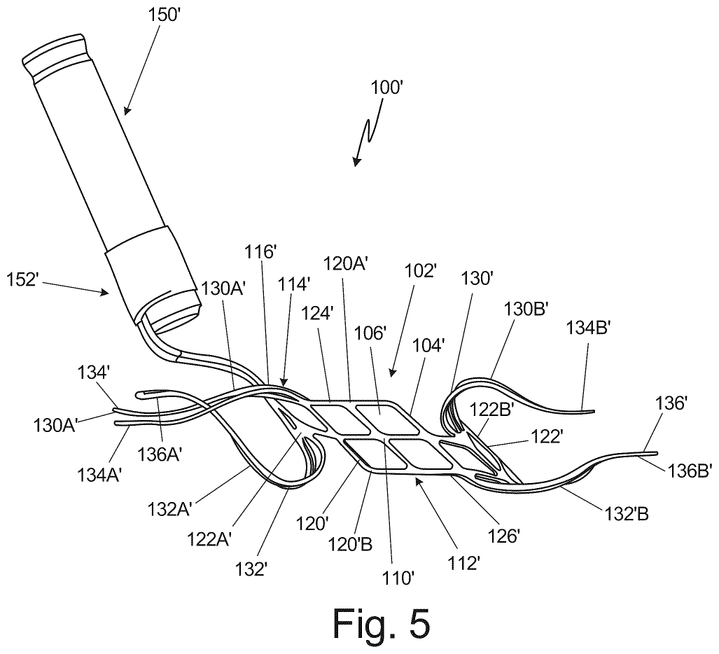

FIG. 5 is a perspective view of a shunt device including a sensor.

### Delivery Catheter 200

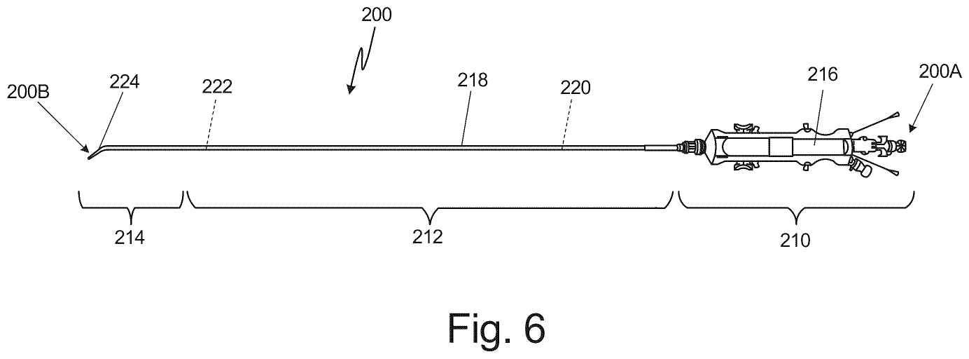

FIG. 6 is a side view of a delivery catheter.

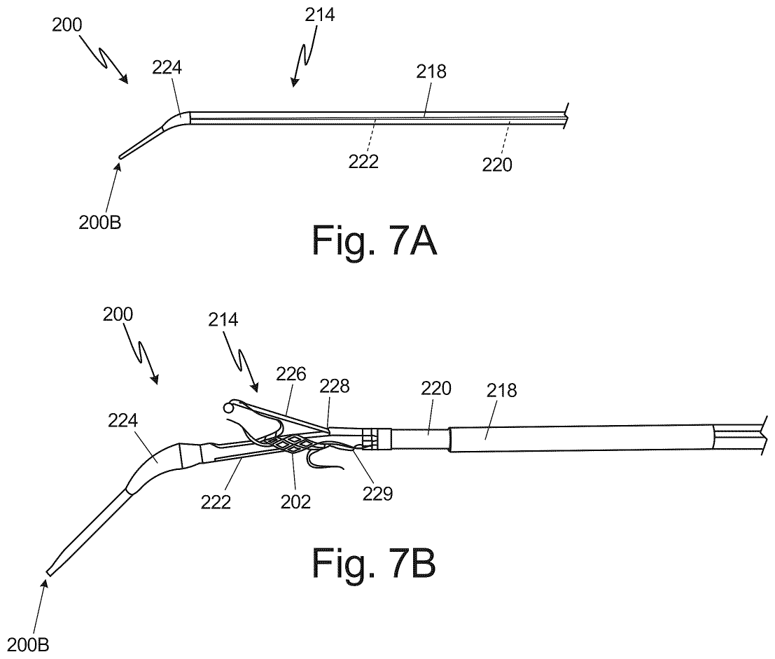

FIG. 7A is a side view of a distal portion of the delivery catheter in a sheathed state.

FIG. 7B is a side view of the distal portion of the delivery catheter in an unsheathed state.

### Delivery Method 300

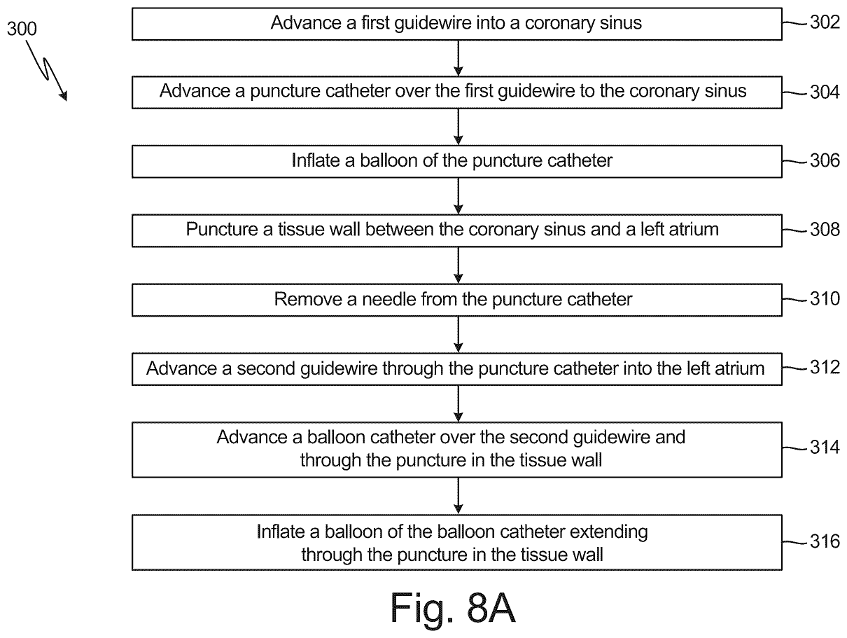

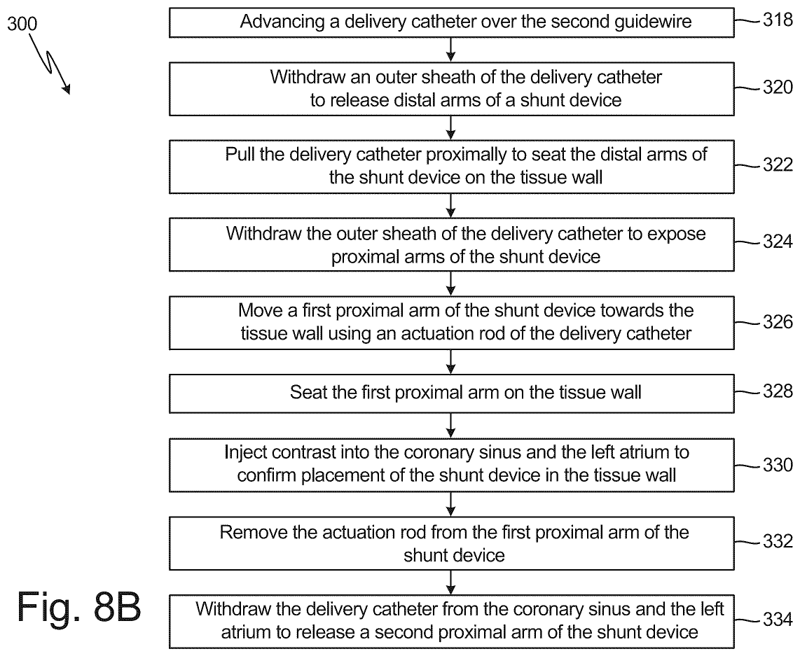

FIG. 8A is a flow chart showing steps for creating a puncture in a tissue wall between a coronary sinus and a left atrium.

FIG. 8B is a flow chart showing steps for implanting a shunt device in the tissue wall between the coronary sinus and the left atrium.

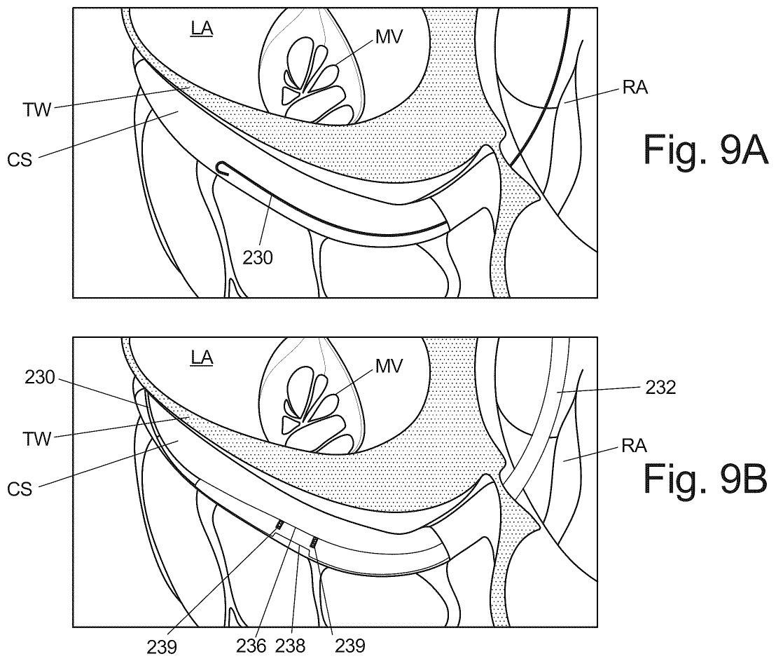

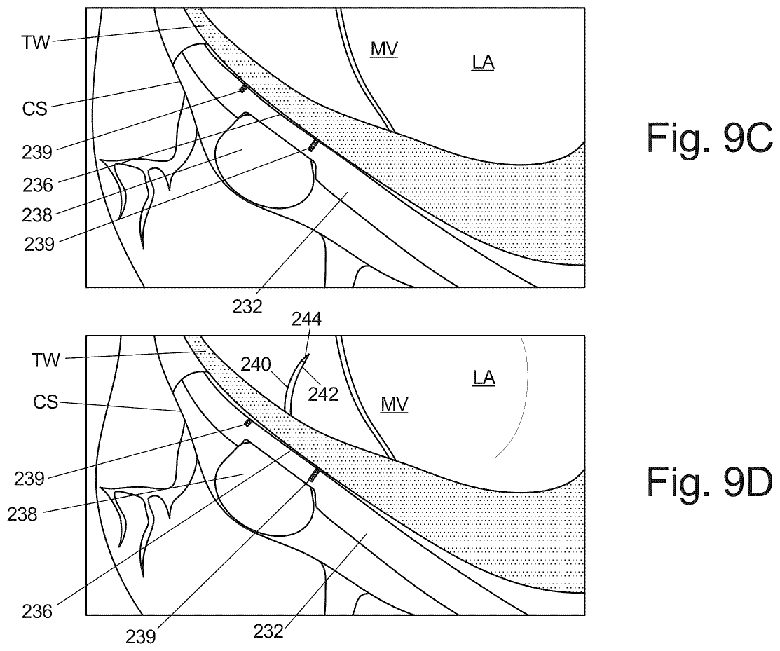

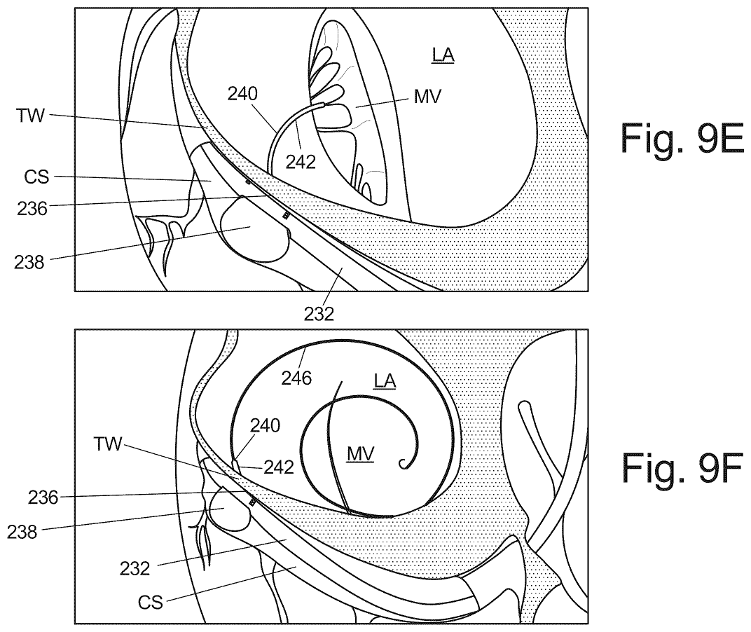

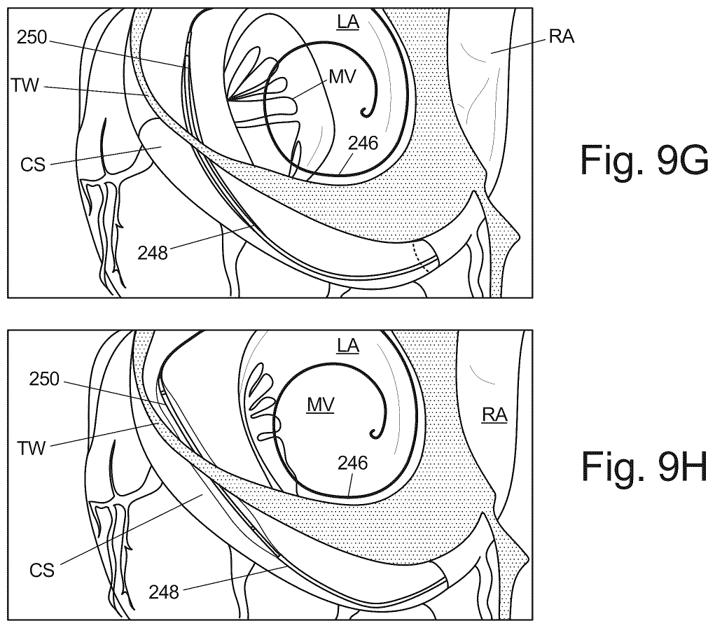

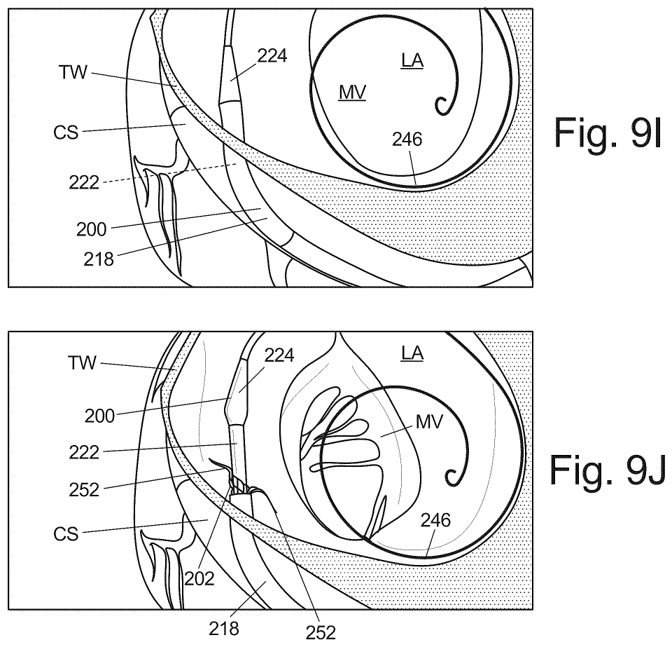

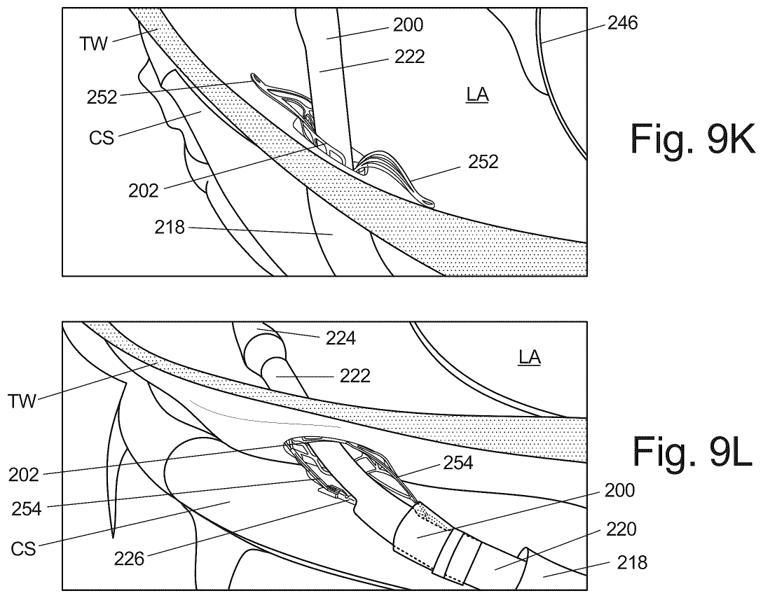

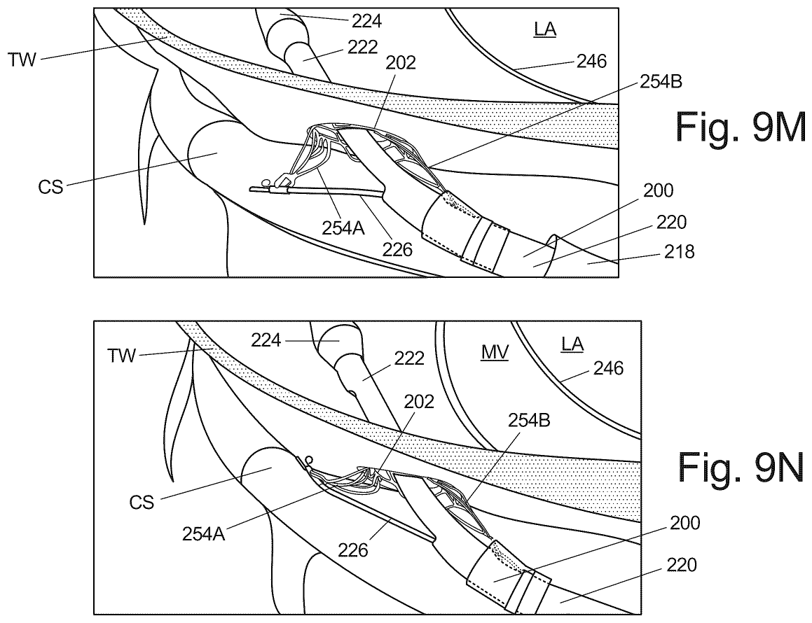

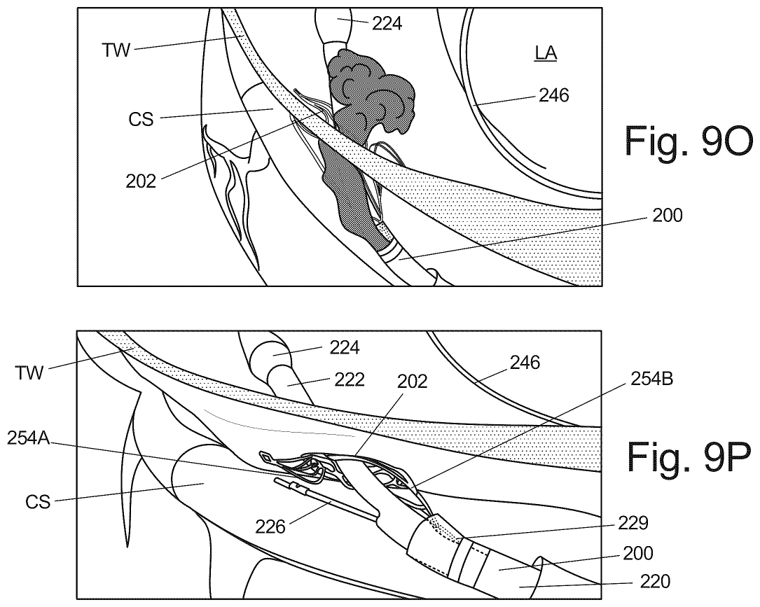

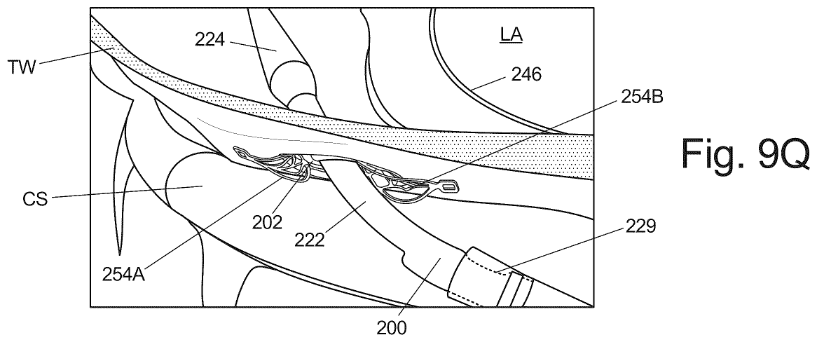

FIGS. 9A-9Q are schematic views showing the steps for implanting a shunt device in the tissue wall between the coronary sinus and the left atrium.

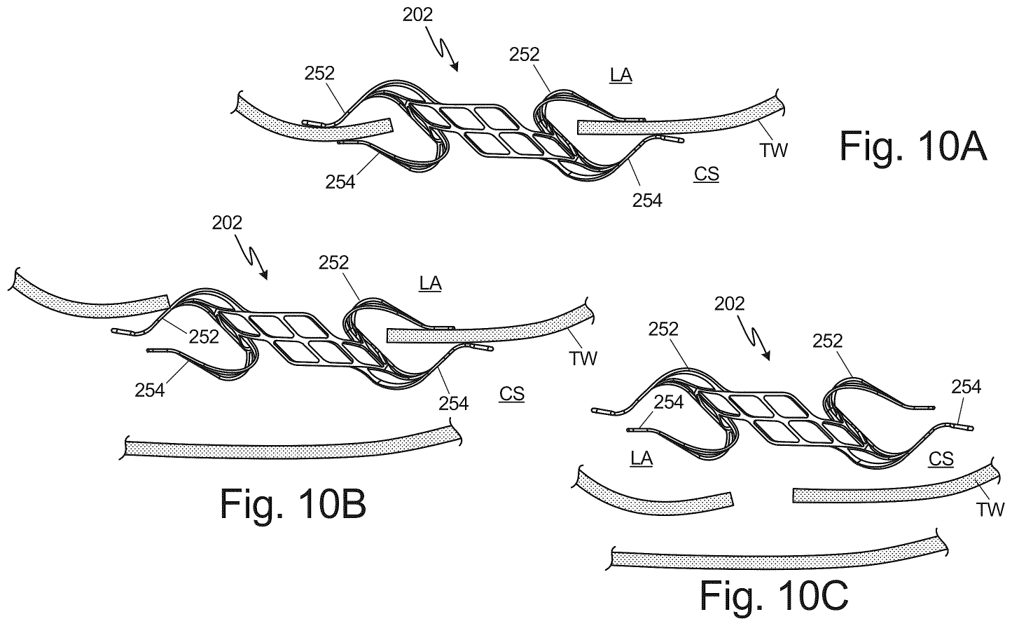

FIG. 10A is a simplified perspective view of the shunt device properly seated between the left atrium and coronary sinus.

FIG. 10B is a simplified perspective view of the shunt device improperly seated between the left atrium and coronary sinus.

FIG. 10C is a simplified perspective view of the shunt device embolized in a left atrium.

### Delivery Device 1400

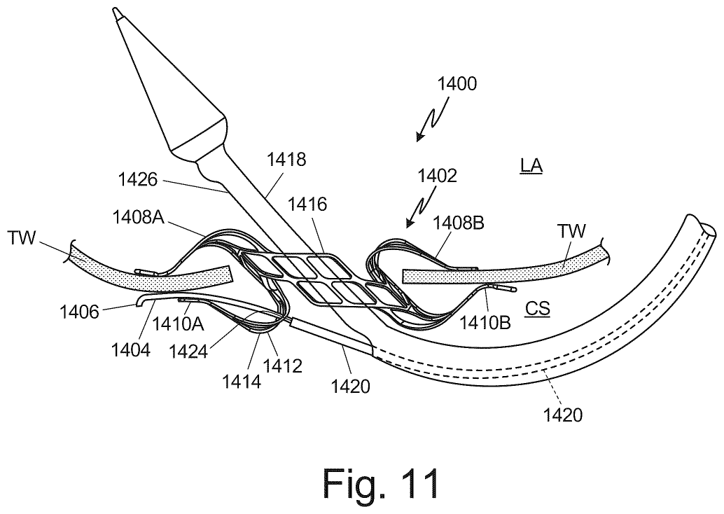

FIG. 11 is perspective view of an implant delivery device including a flexible wire to confirm tissue capture of the shunt device.

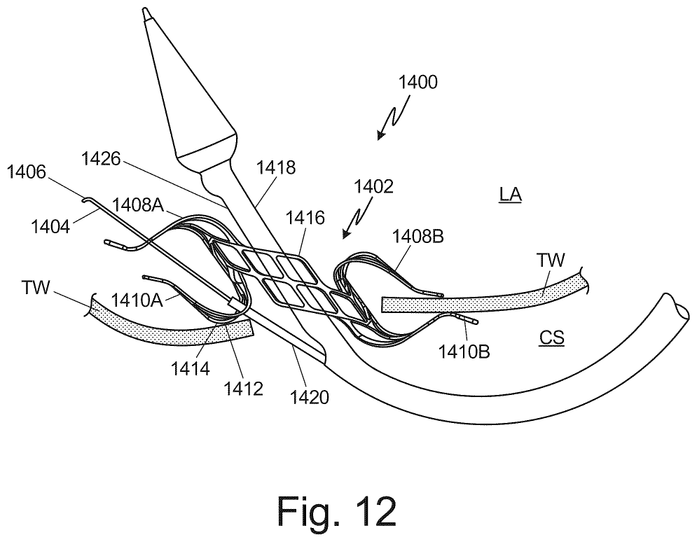

FIG. 12 is a perspective view of the implant delivery device of FIG. 11 illustrating improper seating of the shunt device as confirmed by the flexible wire.

### DETAILED DESCRIPTION

### Anatomy of Heart H and Vasculature V (FIGS. 1-2)

FIG. 1 is a schematic diagram of heart H and vasculature V. FIG. 2 is a cross-sectional view of heart H. FIGS. 1-2 will be described together. FIGS. 1-2 show heart H, vasculature V, right atrium RA, right ventricle RV, left atrium LA, left ventricle LV, superior vena cava SVC, inferior vena cava IVC, tricuspid valve TV (shown in FIG. 1), pulmonary valve PV (shown in FIG. 1), pulmonary artery PA (shown in FIG. 1), pulmonary veins PVS, mitral valve MV, aortic valve AV (shown in FIG. 1), aorta AT (shown in FIG. 1), coronary sinus CS (shown in FIG. 2), thebesian valve BV (shown in FIG. 2), inter-atrial septum IS (shown in FIG. 2), and fossa ovalis FO (shown in FIG. 2).

Heart H is a human heart that receives blood from and delivers blood to vasculature V. Heart H includes four chambers: right atrium RA, right ventricle RV, left atrium LA, and left ventricle LV.

The right side of heart H, including right atrium RA and right ventricle RV, receives deoxygenated blood from vasculature V and pumps the blood to the lungs. Blood flows into right atrium RA from superior vena cava SVC and inferior vena cava IVC. Right atrium RA pumps the blood through tricuspid valve TV into right ventricle RV. The blood is then pumped by right ventricle RV through pulmonary valve PV into pulmonary artery PA. The blood flows from pulmonary artery PA into arteries that delivery the deoxygenated blood to the lungs via the pulmonary circulatory system. The lungs can then oxygenate the blood.

The left side of heart H, including left atrium LA and left ventricle LV, receives the oxygenated blood from the lungs and pumps the blood to the body. Blood flows into left atrium LA from pulmonary veins PVS. Left atrium LA pumps the blood through mitral valve MV into left ventricle LV. The blood is then pumped by left ventricle LV through aortic valve AV into aorta AT. The blood flows from aorta AT into arteries that deliver the oxygenated blood to the body via the systemic circulatory system.

Blood is additionally received in right atrium RA from coronary sinus CS. Coronary sinus CS collects deoxygenated blood from the heart muscle and delivers it to right atrium RA. Thebesian valve BV is a semicircular fold of tissue at the opening of coronary sinus CS in right atrium RA. Coronary sinus CS is wrapped around heart H and runs in part along and beneath the floor of left atrium LA right above mitral valve MV, as shown in FIG. 2. Coronary sinus CS has an increasing diameter as it connects to right atrium RA.

Inter-atrial septum IS and fossa ovalis FS are also shown in FIG. 2. Inter-atrial septum IS is the wall that separates right atrium RA from left atrium LA. Fossa ovalis FS is a depression in inter-atrial septum IS in right atrium RA. At birth, a congenital structure called a foramen ovale is positioned in inter-atrial septum IS. The foramen ovale is an opening in inter-atrial septum IS that closes shortly after birth to form fossa ovalis FS. The foramen ovale serves as a functional shunt in utero, allowing blood to move from right atrium RA to left atrium LA to then be circulated through the body. This is necessary in utero, as the lungs are in a sack of fluid and do not oxygenate the blood. Rather, oxygenated blood is received from the mother. The oxygenated blood from the mother flows from the placenta into inferior vena cava IVC through the umbilical vein and the ductus venosus. The oxygenated blood moves through inferior vena cava IVC to right atrium RA. The opening of inferior vena cava IVC in right atrium RA is positioned to direct the oxygenated blood through right atrium RA and the foramen ovale into left atrium LA. Left atrium LA can then pump the oxygenated blood into left ventricle LV, which pumps the oxygenated blood to aorta AT and the systemic circulatory system. This allows the pulmonary circulatory system to be bypassed in utero. Upon birth, respiration expands the lungs, blood begins to circulate through the lungs to be oxygenated, and the foramen ovale closes to form fossa ovalis FS.

Shunt devices can be positioned in heart H to shunt blood between left atrium LA and right atrium RA. Left atrium LA can experience elevated pressure due to abnormal heart conditions. It has been hypothesized that patients with elevated pressure in left atrium LA may benefit from a reduction of pressure in left atrium LA. Shunt devices can be used in these patients to shunt blood from left atrium LA to right atrium RA to reduce the pressure of blood in left atrium LA, which reduces the systolic preload on left ventricle LV. Reducing pressure in left atrium LA further relieves back-pressure on the pulmonary circulation to reduce the risk of pulmonary edema.

For example, shunt devices can be used to treat patients with heart failure (also known as congestive heart failure). The hearts of patients with heart failure do not pump blood as well as they should. Heart failure can affect the right side and/or the left side of the heart. Diastolic heart failure (also known as heart failure with preserved ejection fraction) refers to heart failure occurring when the left ventricle is stiff (having less compliance), which makes it hard to relax appropriately and fill with blood. This leads to increased end-diastolic pressure, which causes an elevation of pressure in left atrium LA. There are very few, if any, effective treatments available for diastolic heart failure. Other examples of abnormal heart conditions that cause elevated pressure in left atrium LA are systolic dysfunction of the left ventricle and valve disease.

Septal shunt devices (also called inter-atrial shunt devices) are positioned in inter-atrial septum IS to shunt blood directly from left atrium LA to right atrium RA. Typically, septal shunt devices are positioned in fossa ovalis FS, as fossa ovalis FS is a thinner area of tissue in inter-atrial septum IS where the two atria share a common wall. If the pressure in right atrium RA exceeds the pressure in left atrium LA, septal shunt devices can allow blood to flow from right atrium RA to left atrium LA. This causes a risk of paradoxical stroke (also known as paradoxical embolism), as emboli can move from right atrium RA to left atrium LA and then into aorta AT and the systemic circulation.

Shunt devices can also be left atrium to coronary sinus shunt devices that are positioned in a tissue wall between left atrium LA and coronary sinus CS where the two structures are in close approximation. Left atrium to coronary sinus shunt devices move blood from left atrium LA into coronary sinus CS, which then delivers the blood to right atrium RA via thebesian valve BV, the natural orifice of coronary sinus CS. Coronary sinus CS acts as an additional compliance chamber when using a left atrium to coronary sinus shunt device. Left atrium to coronary sinus shunt devices further provide increased protections against paradoxical strokes, as the blood would have to flow retrograde from right atrium RA through coronary sinus CS before entering left atrium LA. Further, left atrium to coronary sinus shunt devices also provide protection against significant right atrium RA to left atrium LA shunting, as again the blood would have to flow retrograde from right atrium RA through coronary sinus CS before entering left atrium LA.

### Shunt Devices 100 and 100′ (FIGS. 3A-5)

FIG. 3A is a perspective view of shunt device 100. FIG. 3B is a side view of shunt device 100. FIG. 4 is a perspective view of shunt device 100 in a collapsed configuration. FIGS. 3A, 3B, and 4 will be described together. Shunt device 100 includes body 102, which is formed of struts 104 and openings 106. Body 102 includes central flow tube 110, flow path 112, and arms 114. Shunt device 100 also includes tissue capture features 116. Central flow tube 110 has side portions 120 (including side portion 120A and side portion 120B), end portions 122 (including end portion 122A and end portion 122B), first axial end 124, and second axial end 126. Arms 114 include distal arms 130 (including distal arm 130A and distal arm 130B) and proximal arms 132 (including proximal arm 132A and proximal arm 132B). Distal arms 130 have terminal ends 134 (including terminal end 134A and terminal end 134B). Proximal arms 132 have terminal ends 136 (including terminal end 136A and terminal end 136B). FIG. 3B further shows gap G, horizontal reference plane HP, perpendicular reference axis RA, central axis CA, tilt angle θ, first angle α, and second angle β.

Shunt device 100 is a cardiovascular shunt. Shunt device 100 is shown in an expanded configuration in FIGS. 3A-3B. Shunt device 100 is formed of a super-elastic material that is capable of being compressed into a catheter for delivery into the body that can then retain its relaxed, or expanded, shape when it is released from the catheter. For example, shunt device 100 can be formed of a shape-memory material, such as nitinol (a nickel titanium alloy). Shunt device 100 is shown in a compressed configuration in FIG. 4. Upon delivery into the body, shunt device 100 will expand back to its relaxed, or expanded, shape. Shunt device 100 can be sterilized before being delivered into the body. Shunt device 100 has body 102 that is formed of interconnected struts 104. Openings 106 in body 102 are defined by struts 104. Body 102 of shunt device 100 is formed of struts 104 to increase the flexibility of shunt device 100 to enable it to be compressed and expanded.

Body 102 includes central flow tube 110 that forms a center portion of shunt device 100. Central flow tube 110 is tubular in cross-section but is formed of struts 104 and openings 106. Central flow tube 110 can be positioned in a puncture or opening in a tissue wall and hold the puncture open. Flow path 112 is an opening extending through central flow tube 110. Flow path 112 is the path through which blood flows through shunt device 100 when shunt device 100 is implanted in the body. Arms 114 extend from central flow tube 110. Arms 114 extend outward from central flow tube 110 when shunt device 100 is in an expanded configuration. Arms 114 hold shunt device 100 in position in the tissue wall when shunt device 100 is implanted in the body.

When shunt device 100 is implanted in the tissue wall between the left atrium and the coronary sinus of the heart, central flow tube 110 holds the puncture open so blood can flow from the left atrium to the coronary sinus through flow path 112. Struts 104 of central flow tube 110 form a lattice or cage of sorts that is sufficient to hold the puncture in the tissue wall open around central flow tube 110. Central flow tube 110 extends from first axial end 124 to second axial end 126. Central flow tube 110 is designed to have an axial length, as measured from first axial end 124 to second axial end 126, that approximates the thickness of the tissue wall between the left atrium and the coronary sinus. When shunt device 100 is implanted in the tissue wall between the left atrium and the coronary sinus, first axial end 124 can be facing the left atrium (i.e., a left atrial side of shunt device 100) and second axial end 126 can be facing the coronary sinus (i.e., a coronary sinus side of shunt device 100). In other examples, the orientation of first axial end 124 and second axial end 126 can be reversed.

Central flow tube 110 has side portions 120 and end portions 122. Side portion 120A and side portion 120B form opposing sides of central flow tube 110. End portion 122A and end portion 122B form opposing ends of central flow tube 110. End portion 122A and end portion 122B each extend between and connect to side portion 120A and side portion 120B to form a generally circular or oval opening that defines flow path 112. Side portions 120 and end portions 122 form a tubular lattice for central flow tube 110. Struts 104 of central flow tube 110 define openings 106 in central flow tube 110. In some examples, openings 106 can be generally parallelogram-shaped. In other examples, openings 106 can be any regular or irregular shape as desired. For example, struts 104 of side portions 120 can form an array of parallelogram-shaped openings 106 in side portions 120. Struts 104 of end portions 122 can form openings 106 in end portions 122. Struts 104 of arms 114 can form openings 106 in arms 114.

As shown in FIG. 3B, central flow tube 110 is angled with respect to horizontal reference plane HP extending through shunt device 100. Horizontal reference plane HP lies generally in the plane of the tissue wall immediately adjacent to shunt device 100 when shunt device 100 is implanted in the tissue wall. End portions 122 are similarly angled with respect to horizontal reference plane HP. Perpendicular reference axis RA, as shown in FIG. 3B, is perpendicular to horizontal reference plane HP. As shown in FIG. 3B, central axis CA is an axis through the center of central flow tube 110 and flow path 112. Central axis CA extends through central flow tube 110 at tilt angle θ with respect to perpendicular reference axis RA. Accordingly, central axis CA defines the angle or tilt of central flow tube 110 with respect to perpendicular reference axis RA (and horizontal reference plane HP). End portions 122 of central flow tube 110 extend parallel to central axis CA.

Arms 114 of shunt device 100 include two distal arms 130 and two proximal arms 132. In some examples, individual ones of distal arms 130 and/or proximal arms 132 can be formed of multiple split arm portions. Arms 114 extend outward from end portions 122 of central flow tube 110 when shunt device 100 is in an expanded configuration. Distal arm 130A is connected to and extends away from end portion 122A, and distal arm 130B is connected to and extends away from end portion 122B. Proximal arm 132A is connected to and extends away from end portion 122A, and proximal arm 132B is connected to and extends away from end portion 122B. When shunt device 100 is implanted in the tissue wall between the left atrium and the coronary sinus, distal arms 130 will be positioned in the left atrium and proximal arms 132 will be positioned in the coronary sinus. Distal arms 130 each have terminal ends 134. Specifically, distal arm 130A has terminal end 134A, and distal arm 130B has terminal end 134B. Proximal arms 132 each have terminal ends 136. Specifically, proximal arm 132A has terminal end 136A, and proximal arm 132B has terminal end 136B.

Distal arms 130 and proximal arms 132 curl outward from end walls 122. As shown in FIG. 3B, each of distal arms 130 and proximal arms 132 has a proximal portion adjacent to central flow tube 110 that forms a shallow curve or arc in a direction away from end walls 122 of central flow tube 110. Each of distal arms 130 and proximal arms 132 flattens out towards respective terminal ends 134 and 136 such that a portion of each of distal arms 130 and proximal arms 132 at or adjacent to the respective terminal end 134 or 136 is generally parallel to horizontal reference plane HP. Accordingly, an axis drawn through terminal end 134A and an axis drawn through terminal end 136B, which are approximated in FIG. 3B as axes in the plane of horizontal reference plane HP for simplicity, can each form first angle α with central axis CA through central flow tube 110. Similarly, an axis drawn through terminal end 134B, and an axis drawn through terminal end 136A, which are approximated in FIG. 3B as axes in the plane of horizontal reference plane HP for simplicity, can each form second angle β with central axis CA through central flow tube 110. Alternatively, distal arms 130 and proximal arms 132 do not flatten out and become parallel to horizontal reference plane HP but instead approach horizontal reference plane HP at an angle and/or have respective terminal ends 134 and 136 that angle away from horizontal reference plane HP. In such examples, first angle α and second angle β are approximations of the central angle for the arcs from end walls 122 to the tissue wall that each respective arm encompasses when shunt device 100 is implanted in the tissue wall. Put more simply, first angle α is the angle between central axis CA and horizontal reference plane HP, and second angle β is the supplementary angle to first angle α. In some examples, first angle α can be less than ninety degrees (<90°) and second angle β can be greater than ninety degrees (>90°). In other examples, first angle α and second angle β can be any suitable combination of angles that add to one hundred eighty degrees (180°). The difference between first angle α and second angle β (and the corresponding curvature of ones of distal arms 130 and proximal arms 132) accommodates for the tilt of central flow tube 110.

As shown in FIG. 3B, distal arm 130A and distal arm 130B extend outwards from central flow tube 110 in opposite directions parallel to horizontal reference plane HP. Distal arm 130A and distal arm 130B can be aligned with each other (i.e., oriented at 180° to each other across central flow tube 110). In some examples, distal arm 130A has a longer length than distal arm 130B. In other examples, distal arm 130A has a shorter length than distal arm 130B. In yet other examples, distal arms 130 can have similar lengths. Proximal arm 132A and proximal arm 132B extend outwards from central flow tube 110 in opposite directions parallel to horizontal reference plane HP. Proximal arm 132A and proximal arm 132B can be aligned with each other (i.e., oriented at 180° to each other across central flow tube 110). In some examples, proximal arm 132A has a shorter length than proximal arm 132B. In other examples, proximal arm 132A has a longer length than proximal arm 132B. In yet other examples, proximal arms 132 can have similar lengths. In some examples, distal arm 130A has generally the same length and shape as proximal arm 132B, and distal arm 130B has generally the same length and shape as proximal arm 132A. In other examples, each of distal arms 130 and proximal arms 132 can have different lengths and shapes, though the overall shape of each arm is similar. As such, shunt device 100 has some degree of inverse symmetry across horizontal reference plane HP, as shown in FIG. 3B.

Shunt device 100 is generally elongated longitudinally but is relatively narrow laterally. Stated another way, distal arms 130 and proximal arms 132 are not annular or circular, but rather extend outward generally in only one plane. As shown in FIG. 3B, shunt device 100 has a generally H-shape when viewing a side of shunt device 100. The elongated shape of shunt device 100 means that when compressed it elongates along a line, as shown in FIG. 4, so as to better fit within a catheter.

Terminal ends 134 of distal arms 130 and terminal ends 136 of proximal arms 132 converge towards one another. Distal arms 130 and proximal arms 132 form two pairs of arms. That is, each of distal arms 130 forms a clamping pair with a corresponding one of proximal arms 132. Distal arm 130A and proximal arm 132A form a first pair of arms extending outward from a first side of central flow tube 110, and terminal end 134A of distal arm 130A converges towards terminal end 136A of proximal arm 132A. Distal arm 130B and proximal arm 132B form a second pair of arms extending outward from a second side of central flow tube 110, and terminal end 134B of distal arm 130B converges towards terminal end 136B of proximal arm 132B. Gap G between terminal ends 134 and terminal ends 136 is sized to be slightly smaller than an approximate thickness of the tissue wall between the left atrium and the coronary sinus, or another tissue wall of interest. This allows distal arms 130 and proximal arms 132 to flex outwards and grip the tissue wall when implanted to help hold shunt device 100 in place against the tissue wall. Thus, a distance corresponding to gap G, as measured once shunt device 100 is implanted, may be slightly different between different clamping pairs of distal arms 130 and proximal arms 132 depending on anatomical variations along the particular tissue wall. Terminal ends 134 of distal arms 130 and terminal ends 136 of proximal arms 132 can also have openings or indentations that are configured to engage a delivery tool to facilitate implantation of shunt device 100, for example actuating rods of a delivery tool. Additionally, terminal ends 134 of distal arms 130 and terminal ends of proximal arms 132 can include locations for radiopaque markers to permit visualization of the positioning of shunt device 100.

When implanted in the tissue wall, distal arms 130 and proximal arms 132 are designed such that the projection of distal arms 130 and proximal arms 132 into the left atrium and the coronary sinus, respectively, is minimized. This minimizes the disruption of the natural flow patterns in the left atrium and the coronary sinus. Shunt device 100 can also be designed so that the profile of proximal arms 132 projecting into the coronary sinus is lower than the profile of distal arms 130 projecting into the left atrium to minimize disruption of the natural blood flow through the coronary sinus and to reduce the potential for proximal arms 132 to block the narrower passage of the coronary sinus.

Tissue capture features 116 can take several different forms. For example, tissue capture features 116 connected to central flow tube 110 at first axial end 124 and/or second axial end 126 can be tabs that extend outward from side portions 120. Tissue capture features 116 connected to arms 114 can be deflectable projections that extend between respective ones of arms 114 and the tissue wall to be compressed back toward the respective arm 114 when shunt device 100 is implanted in the tissue wall. Tissue capture features 116 connected to end portions 122 of central flow tube 110 can be secondary arms associated with one of arms 114. Tissue capture features 116 that are a part of arms 114 themselves can be, e.g., a lengthened portion of one of arms 114, separate split arm portions of one of arms 114, and/or interlacing arms 114. Any one or more of tissue capture features 116 can be incorporated alone or in combination on shunt device 100 to aid in anchoring shunt device 100 to the tissue wall and to prevent displacement of shunt device 100.

FIG. 5 is a perspective view of shunt device 100′ including sensor 150′. Shunt device 100′ includes body 102′, which is formed of struts 104′ and openings 106′. Body 102′ includes central flow tube 110′, flow path 112′, arms 114′. Shunt device 100′ also includes and tissue capture features 116′. Central flow tube 110′ has side portions 120′ (including side portion 120A′ and side portion 120B′), end portions 122′ (including end portion 122A′ and end portion 122B′), first axial end 124′, and second axial end 126′. Arms 114′ include distal arms 130′ (including distal arm 130A′ and distal arm 130B′) and proximal arms 132′ (including proximal arm 132A′ and proximal arm 132B′). Distal arms 130′ have terminal ends 134′ (including terminal end 134A′ and terminal end 134B′). Proximal arms 132′ have terminal ends 136′ (including terminal end 136A′ and terminal end 136B′). Shunt device 100′ further includes sensor 150′ and sensor attachment portion 152′.

Shunt device 100′ includes a similar structure and design to shunt device 100 described above, except shunt device 100′ additionally includes sensor 150′ connected to sensor attachment portion 152′.

As shown in FIG. 5, sensor 150′ can be attached to shunt device 100′ so that sensor 150′ is positioned in the left atrium when shunt device 100′ is implanted in the tissue wall between the left atrium and the coronary sinus of the heart. Accordingly, sensor 150′ can be attached to one of distal arms 130′. Alternatively, sensor 150′ can be attached to shunt device 100′ so that sensor 150′ is positioned in the coronary sinus when shunt device 100′ is implanted in the tissue wall. In such examples, sensor 150′ can be attached to one of proximal arms 132′. In further examples, an additional sensor can be included on shunt device 100′ to position sensors in both the left atrium and the coronary sinus.

Sensor 150′ is attached to shunt device 100′ at sensor attachment portion 152′. Sensor 150′ can be connected to sensor attachment portion 152′ using any suitable attachment mechanism. For example, sensor 150′ and sensor attachment portion 152′ can include complimentary mating features. Sensor attachment portion 152′ can be an extension of one of arms 114′ of shunt device 100′. In some examples, sensor attachment portion 152′ is an extension of distal arm 130A′. In other examples, sensor attachment portion 152′ is an extension of distal arm 130B′ or one of proximal arms 132′. Alternatively, as shown in FIG. 5, sensor attachment portion 152′ can be a separate split arm portion of one of arms 114′. Sensor attachment portion 152′ can be angled away from a horizontal reference plane (not shown) that is in the plane of the tissue wall adjacent to shunt device 100′ when shunt device 100′ is implanted in the tissue wall. That is, sensor attachment portion 152′ can be angled away from the tissue wall.

Sensor 150′ can be a pressure sensor to sense a pressure in the left atrium. In other examples, sensor 150′ can be any sensor to measure a parameter in the left atrium. In yet other examples, sensor 150′ can be any sensor to measure a parameter in the coronary sinus. Sensor 150′ can include a transducer, control circuitry, and an antenna in one example. The transducer, for example a pressure transducer, is configured to sense a signal from the left atrium. The transducer can communicate the signal to the control circuitry. The control circuitry can process the signal from the transducer or communicate the signal from the transducer to a remote device outside of the body using the antenna. Sensor 150′ can include alternate or additional components in other examples. Further, the components of sensor 150′ can be held in a sensor housing that is hermetically sealed.

### Delivery Catheter 200 (FIGS. 6-7B)

FIG. 6 is a side view of delivery catheter 200. FIG. 7A is a side view of distal portion 214 of delivery catheter 200 in a sheathed state. FIG. 7B is a side view of distal portion 214 of delivery catheter 200 in an unsheathed state. FIGS. 6, 7A, and 7B will be discussed together. FIGS. 6-7B show delivery catheter 200. FIG. 7B shows shunt device 202. Delivery catheter 200 includes proximal end 200A, distal end 200B, proximal portion 210, intermediate portion 212, distal portion 214, handle 216, outer sheath 218, inner sheath 220, bridge 222, nosecone 224, actuation rod 226, side opening 228, and notch 229.

Delivery catheter 200 is one example of a delivery catheter that can be used to implant a shunt device into a patient. Delivery catheter 200 as shown in FIGS. 6-7B is used to implant shunt device 202 (shown in FIG. 7B). Delivery catheter 200 can take other forms in alternate examples. Shunt device 202 can have the structure and design of any suitable shunt device, for example shunt device 100 or 100′ as shown in FIGS. 3A-5. Delivery catheter 200 is shown as being configured to implant shunt device 202 without a sensor in the example shown in FIGS. 6-7B. In alternate examples, delivery catheter 200 can be used to implant a shunt device with a sensor, including any needed modifications to accommodate the sensor.

Delivery catheter 200 includes proximal portion 210 adjacent proximal end 200A of delivery catheter 200, intermediate portion 212 extending from proximal portion 210, and distal portion 214 extending from intermediate portion 212 to distal end 200B of delivery catheter 200. Proximal portion 210 includes handle 216, which can be grasped by a physician to control movement of delivery catheter 200. Handle 216 includes a number of ports through which guide wires, tubes, fluids, or other components or elements may be passed.

Intermediate portion 212 extends outward from handle 216 and is a length of catheter that can be moved through a patient. Outer sheath 218 and inner sheath 220 extend outward from handle 216 and form a portion of intermediate portion 212. Outer sheath 218 covers inner sheath 220.

Distal portion 214 extends from intermediate portion 212. Distal portion 214 includes bridge 222 and nosecone 224. Bridge 222 extends from inner sheath 220 towards nosecone 224. Nosecone 224 extends from bridge 222 to distal end 200B of delivery catheter 200. Bridge 222 is configured to hold shunt device 202. As shown in FIG. 7A, when delivery catheter 200 is in a sheathed state, outer sheath 218 will extend over and cover shunt device 202 on bridge 222. As shown in FIG. 7B, when delivery catheter 200 is in an unsheathed state, outer sheath 218 will be pulled back to expose bridge 222 and shunt device 202 on bridge 222. Nosecone 224 extends outward from bridge 222 and helps guide delivery catheter 200 through a patient's vasculature. Actuation rod 226, also called an actuation arm, extends through a lumen in inner sheath 220 and bridge 222. Actuation rod 226 emerges from side opening 228 in bridge 222 and connects to a first proximal arm of shunt device 202. Side opening 228 extends into a body of bridge 222. Notch 229 extends into the body of bridge 222 opposite side opening 228. Notch 229 is configured to seat a second proximal arm of shunt device 202. The second proximal arm can be retained on bridge 222 prior to deployment by a release wire (not shown) extending through a lumen of bridge 222 and through notch 229.

Delivery catheter 200 will be discussed below in more detail with respect to FIGS. 8A-9Q.

### Delivery Method 300 (FIGS. 8A-9Q)

FIG. 8A is a flow chart showing steps for creating a puncture in tissue wall TW between coronary sinus CS and left atrium LA. FIG. 8B is a flow chart showing steps for implanting shunt device 202 in tissue wall TW between coronary sinus CS and left atrium LA. FIGS. 9A-9Q are schematic views showing the steps for implanting shunt device 202 in tissue wall TW between coronary sinus CS and left atrium LA. FIGS. 8A-9Q will be discussed together. FIGS. 8A-8B show method 300. FIG. 8A shows steps 302-316 of method 300. FIG. 8B shows steps 318-334 of method 300.

Step 302 includes advancing guidewire 230 into coronary sinus CS, as shown in FIG. 9A. Guidewire 230 can be inserted using traditional methods. Guidewire 230 is inserted into right atrium RA, through an ostium of coronary sinus CS, and then into coronary sinus CS. Optionally, a catheter having radiopaque markers can be inserted over guidewire 230 and imaging can be done to confirm placement of guidewire 230 in coronary sinus CS. Additionally, contrast can be injected into coronary sinus CS through the catheter to further confirm placement of guidewire 230 in coronary sinus CS. The catheter can then be removed once placement of guidewire 230 in coronary sinus CS is confirmed.

Step 304 includes advancing puncture catheter 232 over guidewire 230 to coronary sinus CS, as shown in FIG. 9B. Puncture catheter 232 is used to puncture tissue wall TW between coronary sinus CS and left atrium LA. Puncture catheter 232 includes catheter body 234 having opening 236 on a first side and balloon 238 on a second side opposite opening 236. Puncture catheter 232 can also include radiopaque markers 239 proximal and distal to opening 236 to confirm placement of puncture catheter 232 in coronary sinus CS. Puncture catheter 232 is advanced into coronary sinus CS so that opening 236 is facing tissue wall TW between coronary sinus CS and left atrium LA. Puncture catheter 232 shown in FIG. 9B is one example of a puncture catheter. In alternate examples, tissue wall TW can be punctured using other puncture catheters or other suitable mechanisms.

Step 306 includes inflating balloon 238 of puncture catheter 232, as shown in FIG. 9C. As balloon 238 is inflated, it will press against coronary sinus CS opposite of tissue wall TW. The inflation of balloon 238 will press puncture catheter 232 against tissue wall TW. Specifically, opening 236 will be pressed against tissue wall TW. Balloon 238 will anchor puncture catheter 232 in position in coronary sinus CS while a puncture is made in tissue wall TW. In alternate examples, any other suitable anchoring mechanism can be used instead of balloon 238. In further examples, step 306 is not needed.

Step 308 includes puncturing tissue wall TW between coronary sinus CS and left atrium LA, as shown in FIG. 9D. Puncture catheter 232 includes puncture arm 240 extending through a lumen in puncture catheter 232. Puncture arm 240 includes sheath 242 and needle 244 positioned in sheath 242 so that it extends out a distal end of puncture sheath 242. Puncture arm 240 can be advanced through puncture catheter 232 and out of opening 236 to puncture through tissue wall TW between coronary sinus CS and left atrium LA.

Puncture catheter 232 should be positioned in coronary sinus CS so that opening 236 of puncture catheter 232 is positioned 2-4 centimeters from the ostium of coronary sinus CS. This will position the puncture through tissue wall TW at the same location. The puncture, and ultimately the placement of shunt device 202 in the puncture, is positioned over the posterior leaflet of mitral valve MV.

Step 310 includes removing needle 244 from puncture catheter 232, as shown in FIG. 9E. Needle 244 can be removed by pulling it proximally through a lumen extending through needle sheath 242 of puncture arm 240. Needle 244 is fully removed from puncture catheter 232, leaving a lumen extending from a proximal end of puncture catheter 232 through a distal end of needle sheath 242.

Step 312 includes advancing guidewire 246 through puncture catheter 232 into left atrium LA, as shown in FIG. 9F. Specifically, guidewire 246 is advanced through a lumen extending through a proximal end of puncture catheter 232 and needle sheath 242 of puncture arm 240. Guidewire 246 is advanced into left atrium LA until it coils in left atrium LA, as shown in FIG. 9F. Once guidewire 246 is fully positioned in left atrium LA, puncture catheter 232 and guidewire 230 can be removed from left atrium LA and coronary sinus CS.

Step 314 includes advancing balloon catheter 248 over guidewire 246 and through the puncture in tissue wall TW, as shown in FIG. 9G. Balloon catheter 248 is advanced through the puncture in tissue wall TW so balloon 250 of balloon catheter 248 is positioned in the puncture in tissue wall TW. Balloon catheter 248 is shown as being a separate device from puncture catheter 232 in the example shown in FIG. 9G. However, in alternate examples, balloon catheter 248 can be inserted through puncture catheter 232 and through the puncture in tissue wall TW.

Step 316 includes inflating balloon 250 of balloon catheter 248 extending through the puncture in tissue wall TW, as shown in FIG. 9H. Balloon 250 extends along a distal portion of balloon catheter 248. As balloon 250 is inflated, it will expand and push open the tissue surrounding the puncture in tissue wall TW. The inflation of balloon 250 will cause the puncture in tissue wall TW to become a wider opening in which a shunt device can be positioned. Balloon 250 can then be deflated and balloon catheter 248 can be removed from left atrium LA and coronary sinus CS.

Step 318 includes advancing delivery catheter 200 over guidewire 246, as shown in FIG. 9I. Delivery catheter 200 has the general structure and design as discussed with reference to FIGS. 6-7B above. Delivery catheter 200 is inserted through coronary sinus CS, through the opening in tissue wall TW, and into left atrium LA. When delivery catheter 200 is properly positioned in tissue wall TW, nosecone 224 will be positioned in left atrium LA, and bridge 222 will extend through tissue wall TW between left atrium LA and coronary sinus CS. Nosecone 224 tapers from a smaller diameter at a distal end to a larger diameter at a proximal end. The taper of nosecone 224 helps to advance nosecone 224 through the opening in tissue wall TW and widens the opening as needed. Bridge 222 holds shunt device 202 (not shown in FIG. 9I) in a collapsed position on bridge 222. Bridge 222 is positioned in tissue wall TW so that shunt device 202 is generally positioned in the opening in tissue wall TW for deployment into the opening.

Step 320 includes withdrawing outer sheath 218 of delivery catheter 200 to release distal arms 252 of shunt device 202, as shown in FIG. 9J. Outer sheath 218 can be withdrawn to expose part of shunt device 202 held on bridge 222 of delivery catheter 200. As outer sheath 218 is withdrawn, distal arms 252 of shunt device 202 will be released and assume their preset shape. Delivery catheter 200 should be positioned in left atrium LA such that when outer sheath 218 is withdrawn to release distal arms 252 of shunts device 202, distal arms 252 of shunt device 202 are positioned in left atrium LA.

Step 322 includes pulling delivery catheter 200 proximally to seat distal arms 252 of shunt device 202 on tissue wall TW, as shown in FIG. 9K. Delivery catheter 200 can be gently pulled proximally to seat distal arms 252 of shunt device 202 on tissue wall TW in left atrium LA. A physician should stop gently pulling on delivery catheter 200 when resistance is sensed, indicating that distal arms 252 have come into contact with tissue wall TW. This will also position a central flow tube of shunt device 202 in the opening in tissue wall TW.

Step 324 includes withdrawing outer sheath 218 of delivery catheter 200 to expose proximal arms 254 of shunt device 202, as shown in FIG. 9L. Outer sheath 218 is withdrawn a set distance to fully expose shunt device 202, including proximal arms 254 of shunt device 202. Delivery catheter 200 should be positioned in left atrium LA, tissue wall TW, and coronary sinus CS so that proximal arms 254 will be positioned in coronary sinus CS when outer sheath 218 is withdrawn. Proximal arms 254 are constrained on bridge 222 of delivery catheter 200 and will not automatically assume their preset shape when outer sheath 218 is withdrawn.

Step 326 includes moving first proximal arm 254A of shunt device 202 towards tissue wall TW using actuation rod 226 of delivery catheter 200, as shown in FIG. 9M. Actuation rod 226 extends through a lumen in delivery catheter 200 and can be actuated forward to move first proximal arm 254A towards tissue wall TW.

Step 328 includes seating first proximal arm 254A on tissue wall TW, as shown in FIG. 9N. Actuation rod 226 of delivery catheter 200 is actuated fully outward to seat first proximal arm 254A on tissue wall TW. When first proximal arm 256A is seated on tissue wall TW, it will be positioned in coronary sinus CS.

Step 330 includes confirming placement of shunt device 202 in tissue wall TW. FIG. 9O illustrates a known method for confirming tissue confirmation, which includes injecting a contrast agent through a lumen extending through delivery catheter 200. The contrast agent can move through coronary sinus CS and left atrium LA. The contrast will highlight shunt device 202 under fluoroscopy to confirm proper placement of distal arms 252 and first proximal arm 254A of shunt device 202 on tissue wall TW. An alternative method for confirming tissue capture is discussed below.

Step 332 includes removing actuation rod 226 from first proximal arm 254A of shunt device 202, as shown in FIG. 9P. Actuation rod 226 can be held on and removed from first proximal arm 254A using any suitable mechanism. In the example shown in FIG. 9P, a release wire holds actuation rod 226 on first proximal arm 254A. The release wire can be withdrawn proximally to disconnect release wire from first proximal arm 254A. Actuation rod 226 can then be pulled proximally through a lumen of delivery catheter 200 to remove actuation rod 226 from coronary sinus CS.

Step 334 includes withdrawing delivery catheter 200 from coronary sinus CS and left atrium LA to release second proximal arm 254B of shunt device 202, as shown in FIG. 9Q. Second proximal arm 254B is held in place on bridge 222 in notch 229 formed in bridge 222. As delivery catheter 200 is withdrawn, second proximal arm 254B will be released from notch 229 in bridge 222 and take its preset shape. Specifically, second proximal arm 254B will seat upon tissue wall TW as it takes its preset shape. Second proximal arm 245B will be positioned in coronary sinus CS. After second proximal arm 254B is seated on tissue wall TW, shunt device 202 will be fully deployed in tissue wall TW, as shown in FIG. 10A. Delivery catheter 200 and guidewire 246 can then be removed from left atrium LA and coronary sinus CS.

Method 300 is one example of a method that can be used to implant shunt device 202 in tissue wall TW between left atrium LA and coronary sinus CS. Method 300 can include fewer, more, or different steps in alternate examples. Further, puncture catheter 232 and delivery catheter 200 are shown as being separate catheters in the example shown in FIGS. 9A-9Q but can be a single catheter in alternate examples.

Shunt devices must be anchored in place to avoid displacement during normal heart rhythms. Techniques are needed to confirmed proper placement of shunt devices during implantation.

FIG. 10A is a simplified perspective view of shunt device 202 properly seated between left atrium LA and coronary sinus CS. FIG. 10B is a simplified perspective view of shunt device 202 improperly seated between left atrium LA and coronary sinus CS. FIG. 10C is a simplified perspective view of shunt device 202 embolized in left atrium LA. FIGS. 10A-10B will be discussed together. FIGS. 10A-10B show shunt device 202, including distal arms 252 and proximal arms 254. FIGS. 10A-10B further show left atrium LA, coronary sinus CS, and tissue wall TW.

FIG. 10A shows shunt device 202 properly seated in tissue wall TW between left atrium LA and coronary sinus CS. As illustrated, distal arms 252 engage tissue wall TW and are positioned in left atrium LA, and proximal arms 254 engage tissue wall TW and are positioned in coronary sinus CS. During deployment, one or more distal arms 252 or proximal arms 254 can be mis-seated. In one example, shunt device 202 could be improperly seated such that one or more of distal arms 252 is positioned in coronary sinus CS rather than in left atrium LA, as illustrated in FIG. 10B. For example, during an implantation procedure (e.g., during step 322 of method 300 as shown in FIG. 8B), a physician may pull delivery catheter 200 back too hard after distal arms 252 are released, causing all or a part of shunt device 202 to be pulled into coronary sinus CS. In another example, shunt device 202 could be improperly seated such that the entirety of shunt device 202 is located in left atrium LA, as illustrated in FIG. 10C. For example, during an implantation procedure (e.g., during step 322 of method 300 as shown in FIG. 8B), a physician may not pull delivery catheter 200 back far enough after distal arms 252 are released, so one or more of proximal arms 254 may be released in or pushed through to left atrium LA, causing shunt device 202 to embolize. Confirming tissue capture between the arms of a shunt device helps a physician to determine when it is safe to release the shunt device. As such, confirming proper seating of the shunt device during and/or following delivery also helps reduce the risk of embolization and/or need for redeployment. The present disclosure includes a soft tip probe for confirming tissue capture and ensuring proper placement of a shunt device.

### Delivery Device 1400

FIG. 11 is perspective view of implant delivery device 1400 for delivering and placing shunt device 1402 within a human body and confirming tissue capture of shunt device 1402 with a soft tip probe or flexible wire 1404. FIG. 12 is a perspective view of implant delivery device 1400 illustrating improper seating of shunt device 1402 as confirmed by flexible wire 1404. FIGS. 11 and 12 will be discussed together. Delivery device 1400, shunt device 1402, flexible wire 1404, tip 1406, distal arms 1408A and 1408B, proximal arms 1410A and 1410B, struts 1412, opening 1414, central flow tube 1416, bridge 1418, tube 1420, distal end 1424, side opening 1426, tissue wall TW, coronary sinus CS, and left atrium LA are shown.

Delivery device 1400 is one example of delivery catheter 200 shown in FIGS. 6-7C that can be used to deliver shunt device 1402 and confirm placement of shunt device 1402 in tissue wall TW. Shunt device 1402 can be deployed as described in method 300 shown in FIGS. 8A, 8B, and 9A-9Q and described with respect thereto with a modification of the step of contrast delivery for tissue capture confirmation shown in FIG. 9O.

Shunt device 1402 can be substantially the same as or similar to shunt devices 100 and 100′ illustrated in FIGS. 3A, 3B, 4, and 5 and described with respect thereto. Shunt device 1402 includes distal arms 1408A and 1408B, proximal arms 1410A and 1410B, struts 1412, opening 1414, and central flow tube 1416. Struts 1412 form a portion of proximal arm 1410A and define opening 1414. Opening 1414 is configured to receive flexible wire 1404 and allow for translation of flexible wire 1404 through proximal arm 1410A. In some examples, shunt device 1402 can include a sensor (e.g., sensor 150′ illustrated in FIG. 3) attached to any one of distal arms 1408A and 1408B or proximal arms 1410A and 1410B.

Delivery device 1400 includes bridge 1418. Bridge 1418 can be substantially similar to bridge 222 of delivery catheter 200 shown in FIG. 7B, with the addition of flexible wire 1404. Bridge 1418 includes a number of lumens extending through it to receive wires, tubes, fluids, or other suitable components. Tube 1420 with distal end 1424 extends through one lumen in bridge 1418. Tube 1420 extends lengthwise through bridge 1418 and into side opening 1426 formed in bridge 1418. Tube 1420 is open at distal end 1424. Flexible wire 1404 is slidably received in a lumen extending through tube 1420. Flexible wire 1404 can extend outward from distal end 1424.

Side opening 1426 can be substantially the same as side opening 228 as described with respect to FIG. 7B. Side opening 1426 cuts into the body of bridge 1418 and extends lengthwise along the side of bridge 1418. Side opening 1426 is positioned adjacent to tube 1420. Side opening 1426 can be configured to seat a body portion of shunt device 1402 for delivery into a human body. In some examples, side opening 1426 can be configured to seat a sensor of shunt device 1402 (i.e., sensor 150′ illustrated in FIG. 3).

Tube 1420 extends lengthwise through at least a portion of bridge 1418. Tube 1420 can extend through the same lumen configured for delivery of a contrast agent in bridge 222 of delivery catheter 200 shown in FIG. 7B. The lumen through which tube 1420 extends can be one of a plurality of lumens disposed in bridge 1418 as previously described with respect to bridge 222 of delivery catheter 200. Tube 1420 is a hollow tube extending into side opening 1426. In some examples, tube 1420 can be co-molded with bridge 1418.

Tube 1420 is open at distal end 1424. Tube 1420 is configured to slidably receive flexible wire 1404. Tube 1420 has an inner diameter greater than an outer diameter of flexible wire 1404, thereby allowing a user to advance and retract flexible wire 1404 within tube 1420. Flexible wire 1404 can be fully removed from tube 1420.

In some examples, tube 1420 can be used to deliver a contrast agent to a region of interest during or following delivery of implant device 1402 to confirm tissue capture by shunt device 1402, as disclosed in method step 330 and illustrated in FIG. 9O. The contrast agent can be provided to tube 1420 when flexible wire 1404 is removed from tube 1420. Tube 1420 can have an inner diameter designed to promote the flow of the contrast agent during injection without requiring application of excessive force by the user. The inner diameter of tube 1420 can be, for example, approximately 0.042 in. (1.07 mm) to promote the flow of the contrast agent during injection without requiring application of excessive force by the user and without generating excessive exit pressure, which could damage tissue wall TW. Tube 1420 can be, for example, a polyamide braid or other flexible material known in the art suitable for delivering a contrast agent. In an alternate example, tube 1420 can be fully removed from delivery device 1400 and contrast can be delivered through the lumen through which tube 1420 extended.

Tube 1420 can be positioned external to central flow tube 1416 through proximal arm 1410A of shunt device 1402 adjacent to central flow tube 1416. Tube 1420 is spaced apart from distal arm 1408A such that tube 1420 is separated from distal arm 1408A upon delivery of shunt device 1402. Tube 1420 can be positioned in proximal arm 1410A of shunt device 1402 during assembly. Tube 1420 can be disposed through opening 1414 between struts 1412 of proximal arm 1410A. Tube 1420 is positioned to direct flexible wire 1404 toward tissue wall TW of coronary sinus CS when shunt device 1402 is properly seated. Opening 1414 can be centrally located in proximal arm 1410A adjacent to central flow tube 1416. In alternative examples, opening 1414 can be located at any position of proximal arm 1410A suitable for directing flexible wire 1404 toward tissue wall TW of coronary sinus CS. Struts 1412 maintain the position of tube 1422 during delivery of shunt device 1402. The opening at distal end 1424 of tube 1420 can be disposed adjacent to central flow tube 1416 to direct the contrast agent between proximal arm 1410A and distal arm 1408B.

Flexible wire 1404 is slidably received in tube 1420 and configured to be advanced and retracted through tube 1420 and along side opening 1426. Flexible wire 1404 extends outward from distal end 1424 into side opening 1426 external to bridge 1418. Flexible wire includes tip 1406 at a distal end configured to deflect upon contact with tissue wall TW. Flexible wire 1404 is disposed external to or outside of central flow tube 1416 and is disposed through opening 1414 of proximal arm 1410A adjacent to central flow tube 1416. As previously discussed, opening 1414 can be centrally located in proximal arm 1410A adjacent to central flow tube 1416. In alternative examples, opening 1414 can be located at any position of proximal arm 1410A suitable for directing flexible wire 1404 toward tissue wall TW. Struts 1412 maintain the position of flexible wire 1404 during delivery of shunt device 1402. Flexible wire 1404 can be positioned in proximal arm 1410A of shunt device 1402 during assembly. For example, bridge 1418, shunt device 1402, and flexible wire 1404 can be enclosed in a sheath (e.g., outer sheath 218 illustrated in FIG. 7B) prior to delivery of shunt device 1402 and flexible wire 1404 can be retained in opening 1414 of proximal arm 1410A.

Flexible wire 1404 can be, for example, a ZIPwire™ having a nitinol core or other suitable guide wire material known in the art. Flexible wire 1404 can be radiopaque such that flexible wire can be visible via x-rays or fluoroscopy. Tip 1406 can be a soft tip J-hook, as known in the art, configured to deflect upon contact with tissue wall TW and prevent puncture of tissue wall TW. Flexible wire 1404 has sufficient rigidity to enable tactile detection of deflection of flexible wire 1404 by a user.

A proximal end (not shown) of flexible wire 1404 opposite tip 1406 is configured to be manipulated by the user (e.g., at handle 216 illustrated in FIG. 7B) to advance and retract flexible wire 1404 to determine proper placement or tissue capture of shunt device 1402. Flexible wire 1404 can be used to confirm tissue capture by tactile sensation, imaging, or a combination thereof. During or following deployment of shunt device 1402, the user can feed flexible wire 1404 through tube 1420, thereby advancing flexible wire 1404 through tube 1420 and proximal arm 1410A of shunt device 1402. Advancement of flexible wire 1404 can be imaged via fluoroscopy, allowing the user to view the advancement and location of flexible wire 1404 with respect to proximal arm 1410A and distal arm 1408A. The user can push and/or twist flexible wire 1404 if resistance is felt to cause flexible wire 1404 to deflect. The tactile sensation of resistance followed by further advancement of flexible wire 1404 with reduced or no resistance may confirm proper placement of shunt device 1402 as flexible wire 1404 hits tissue wall TW, deflects, and advances along tissue wall TW while remaining positioned in coronary sinus CS (e.g., has not crossed into left atrium LA). Proper placement of shunt device 1402 can be further confirmed via fluoroscopy. Imaging showing bending of flexible wire 1404 adjacent to a terminal end of proximal arm 1410A and/or a separation between flexible wire 1404 and distal arm 1408A can confirm proper positioning of shunt device 1402. FIG. 11 illustrates a slight bend in flexible wire 1404 adjacent to central flow tube 1416 and illustrates flexible wire 1404 extending adjacent to the terminal end of proximal arm 1410A indicating translation of flexible wire 1404 along tissue wall TW.

Flexible wire 1404 can also be used to confirm improper positioning of shunt device 1402. Flexible wire 1404 can be used to confirm improper seating of shunt device 1402 by tactile sensation, imaging, or a combination thereof. During or following deployment of shunt device 1402, the user can feed flexible wire 1404 through tube 1420, thereby advancing flexible wire through tube 1420 and proximal arm 1410A of shunt device 1402. Advancement of flexible wire 1404 can be imaged via fluoroscopy, allowing the user to view the advancement and location of flexible wire 1404 with respect to proximal arm 1410A and distal arm 1408A. An absence of a tactile sensation of resistance may confirm improper placement of shunt device 1402 as flexible wire 1404 advances unimpeded between proximal arm 1410A and distal arm 1408A, indicating the absence of tissue wall TW. Improper placement of shunt device 1402 can be further confirmed via fluoroscopy. Imaging showing flexible wire 1404 extending substantially straight from tube 1420 and/or flexible wire 1404 at a location of distal arm 1408A or crossing distal arm 1408A can confirm improper positioning shunt device 1402. FIG. 12 illustrates flexible wire 1404 extending substantially straight from tube 1420 and crossing distal arm 1408A indicating improper positioning of shunt device 1402.

Flexible wire 1404 can be used to confirm tissue capture of shunt device 1402 without the use of a contrast agent in patients for which use of contrast agents is contraindicated. Flexible wire 1404 is designed to allow a user to probe the region in which shunt device 1402 is deployed using tactile sensation and imaging to confirm tissue capture of shunt device 1402. Flexible wire 1404 can be used in combination with the injection of a contrast agent if necessary or desired to further confirm tissue capture. Flexible wire 1404 is removed from tube 1420 prior to injection of the contrast agent into tube 1420.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Possible Embodiments

The following are non-exclusive descriptions of possible embodiments of the present invention.

A device for delivering a shunt device within a human body, the shunt device having a central flow tube, a proximal arm, and a distal arm, the proximal arm and distal arm configured to capture a tissue wall therebetween. The delivery device includes a catheter that includes a tube and a side opening. The delivery device further includes a flexible wire slidably receivable in the tube and extendable into the side opening. The flexible wire includes a tip at a distal end configured to deflect upon contact with human tissue. The flexible wire is disposed outside of the central flow tube of the shunt device.

The device of the preceding paragraph can optionally include, additionally and/or alternatively, any one or more of the following features, configurations and/or additional components:

In an embodiment of the foregoing device, the flexible wire can be disposed through an opening in the proximal arm of the shunt device.

In an embodiment of any of the foregoing devices, the catheter, the shunt device, and the flexible wire can be enclosed in a sheath prior to delivery of the shunt device, and wherein the flexible wire can be retained in the opening of the proximal arm of the shunt device.

In an embodiment of any of the foregoing devices, the flexible wire can be configured to be advanced and retracted through the tube.

In an embodiment of any of the foregoing devices, the flexible wire can include a proximal end configured to be manipulated by a user to advance and retract the flexible wire.

In an embodiment of any of the foregoing devices, the flexible wire can have sufficient rigidity to enable tactile detection, by a user, of deflection of the flexible wire.

In an embodiment of any of the foregoing devices, the flexible wire can be radiopaque.

In an embodiment of any of the foregoing devices, the flexible wire can include a nitinol core.

In an embodiment of any of the foregoing devices, the distal end of the flexible wire can include a J-hook.

In an embodiment of any of the foregoing devices, the flexible wire can be removable from the tube.

In an embodiment of any of the foregoing devices, the tube can be configured to deliver a contrast agent.

In an embodiment of any of the foregoing devices, the tube can include a braided tube co-molded with the catheter and extending into the side opening.

In an embodiment of any of the foregoing devices, the braided tube can have an opening at a distal end configured to deliver the contrast agent.

In an embodiment of any of the foregoing devices, the opening can be disposed adjacent to the central flow tube of the shunt device.

In an embodiment of any of the foregoing devices, the flexible wire can be spaced apart from the distal arm when the shunt device can be properly positioned having the distal arm on a first side of the tissue wall and the proximal arm on a second side of the tissue wall.

In an embodiment of any of the foregoing devices, the shunt device can be a blood flow shunt.

In an embodiment of any of the foregoing devices, the flexible wire can extend through an opening of the distal arm when the shunt device is improperly positioned having the distal arm and the proximal arm both positioned on one side of the tissue wall.

A method of detecting tissue capture of a shunt device, the shunt device having a central flow tube, a proximal arm, and a distal arm, the proximal arm and distal arm configured to capture a tissue wall therebetween. The method includes advancing a flexible wire through a tube of a catheter and the proximal arm of the shunt device, and imaging, by fluoroscopy, the advancement of the flexible wire.

The method of the preceding paragraph can optionally include, additionally and/or alternatively, any one or more of the following features, configurations, additional components, and/or steps:

An embodiment of the foregoing method can further include advancing the flexible wire along the tissue wall.

In an embodiment of any of the foregoing methods, the flexible wire can include a proximal end configured to be manipulated by a user to advance and retract the flexible wire.

In an embodiment of any of the foregoing methods, the shunt device can be a blood flow shunt positioned in the tissue wall between a coronary sinus and a left atrium in a human body.

In an embodiment of any of the foregoing methods, the flexible wire can be radiopaque.

An embodiment of any of the foregoing methods can further include confirming proper positioning of the shunt device with the distal arm on a first side of the tissue wall and the proximal arm on a second side of the tissue wall by imaging the flexible wire extending adjacent to a terminal end of the proximal arm.

An embodiment of any of the foregoing methods can further include confirming proper positioning of the shunt device with the distal arm on a first side of the tissue wall and the proximal arm on a second side of the tissue wall by imaging a separation between the flexible wire and the distal arm of the shunt device.

An embodiment of any of the foregoing methods can further include confirming proper positioning of the shunt device with the distal arm on a first side of the tissue wall and the proximal arm on a second side of the tissue wall by imaging bending of the flexible wire adjacent to the central flow tube.

An embodiment of any of the foregoing methods can further include confirming improper positioning of the shunt device with the distal arm and the proximal arm both on one side of the tissue wall by imaging the flexible wire at a location of the distal arm.

An embodiment of any of the foregoing methods can further include confirming improper positioning of the shunt device with the distal arm and the proximal arm both on one side of the tissue wall by imaging the flexible wire extending substantially straight from the tube.

In an embodiment of any of the foregoing methods, advancing the flexible wire can include pushing or twisting the flexible wire when resistance is felt by the user.

In an embodiment of any of the foregoing methods, the flexible wire can include a tip at a distal end configured to deflect upon contact with human tissue.

In an embodiment of any of the foregoing methods, the distal end can include a J-hook.

In an embodiment of any of the foregoing methods, the flexible wire can include a nitinol core.

An embodiment of any of the foregoing methods can further include confirming proper positioning of the shunt device with the distal arm on a first side of the tissue wall and the proximal arm on a second side of the tissue wall by detecting, via tactile sensation, resistance to advancement of the flexible wire followed by advancement of the flexible wire with reduced resistance.

An embodiment of any of the foregoing methods can further include advancing the flexible wire through or across the distal arm when the shunt device is improperly positioned with the distal arm and the proximal arm both on one side of the tissue wall.

An embodiment of any of the foregoing methods can further include confirming improper positioning of the shunt device with the distal arm and the proximal arm both on one side of the tissue wall by imaging advancement of the flexible wire through or across the distal arm of the shunt device, wherein the flexible wire can be radiopaque.

An embodiment of any of the foregoing methods can further include removing the flexible wire from the tube.

An embodiment of any of the foregoing methods can further include injecting a contrast agent through the tube and imaging a region including the shunt device and the contrast agent.

In an embodiment of any of the foregoing methods, the tube can include a braided tube extending from a side opening of the catheter.

In an embodiment of any of the foregoing methods, the braided tube can be co-molded with the catheter.

In an embodiment of any of the foregoing methods, the braided tube can have an opening configured to deliver the contrast agent at a distal end.

In an embodiment of any of the foregoing methods, the opening can be disposed adjacent to the central flow tube of the shunt device.

A device for delivering a shunt device within a human body, the shunt device having a central flow tube, a proximal arm, and a distal arm, the proximal arm and distal arm configured to capture a tissue wall therebetween. The delivery device includes a catheter including a tube extending outward from the catheter, the tube having an open distal end disposed adjacent to and outside of the central flow tube of the shunt device. The delivery device further includes a flexible wire slidably receivable in the tube, the flexible wire including a tip at a distal end configured to deflect upon contact with human tissue.

The device of the preceding paragraph can optionally include, additionally and/or alternatively, any one or more of the following features, configurations and/or additional components:

In an embodiment of the foregoing device, the flexible wire can be disposed through an opening in the proximal arm.

In an embodiment of any of the foregoing devices, the catheter, the shunt device, and the flexible wire can be enclosed in a sheath prior to delivery of the shunt device, and wherein the flexible wire can be retained in the opening of the proximal arm.

In an embodiment of any of the foregoing devices, the flexible wire can be configured to be advanced and retracted through the tube.

In an embodiment of any of the foregoing devices, the flexible wire can include a proximal end configured to be manipulated by a user to advance and retract the flexible wire.

In an embodiment of any of the foregoing devices, the flexible wire can have sufficient rigidity to enable tactile detection, by a user, of deflection of the flexible wire.

In an embodiment of any of the foregoing devices, the flexible wire can be radiopaque.

In an embodiment of any of the foregoing devices, the flexible wire can include a nitinol core.

In an embodiment of any of the foregoing devices, the distal end of the flexible wire can include a J-hook.

In an embodiment of any of the foregoing devices, the flexible wire can be removable from the tube.

In an embodiment of any of the foregoing devices, the tube can be configured to deliver a contrast agent.

In an embodiment of any of the foregoing devices, the tube can include a braided tube co-molded with the catheter.

In an embodiment of any of the foregoing devices, the flexible wire can be spaced apart from the distal arm when the shunt device is properly positioned having the distal arm on a first side of the tissue wall and the proximal arm on a second side of the tissue wall.

In an embodiment of any of the foregoing devices, the shunt device can be a blood flow shunt.

In an embodiment of any of the foregoing devices, the flexible wire can extend through an opening of the distal arm when the shunt device is improperly positioned having the distal arm and the proximal arm both positioned on one side of the tissue wall.

In an embodiment of any of the foregoing devices, the catheter further can include a side opening extending lengthwise and disposed adjacent to the tube.

In an embodiment of any of the foregoing devices, the catheter can be disposed through the central flow tube of the shunt device.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A device for delivering a shunt device within a human body, the shunt device having a central flow tube, a proximal arm, and a distal arm, the proximal arm and distal arm configured to capture a tissue wall therebetween, the device comprising:
a catheter sized to deliver the shunt device, the catheter comprising a side opening; and:
a flexible wire advanceable from the side opening, the flexible wire having a tip at a distal end for deflecting upon contact with human tissue;
wherein the flexible wire is made from nitinol.

2. The device of claim 1, wherein the catheter further comprises a sheath configured to enclose the shunt device and the flexible wire prior to delivery of the shunt device, and wherein the flexible wire is received in an opening of the proximal arm of the shunt device.

3. The device of claim 1, wherein the flexible wire comprises a proximal end configured to be manipulated by a user to advance and retract the flexible wire, and wherein the flexible wire has sufficient rigidity to enable tactile detection, by a user, of deflection of the flexible wire.

4. The device of claim 1, wherein the flexible wire is radiopaque.

5. The device of claim 1, wherein the distal end of the flexible wire comprises a J-hook.

6. The device of claim 1, wherein the catheter further comprises a tube extending into the side opening, wherein the flexible wire is slidably received in the tube.

7. The device of claim 6, wherein the tube comprises a braided tube co-molded with the catheter, wherein the braided tube has an opening at a distal end configured to deliver a contrast agent, and wherein the opening is disposed adjacent to the central flow tube of the shunt device when the shunt device is seated.

8. The device of claim 1, wherein the flexible wire is configured to be spaced apart from the distal arm when the shunt device is properly positioned having the distal arm on a first side of the tissue wall and the proximal arm on a second side of the tissue wall.

9. A device for delivering a shunt device within a human body, the shunt device having a central flow tube, a proximal arm, and a distal arm, the proximal arm and distal arm configured to capture a tissue wall therebetween, the device comprising:
a catheter sized to deliver the shunt device, the catheter comprising a side opening; and:
a flexible wire advanceable from the side opening, the flexible wire having a tip at a distal end for deflecting upon contact with human tissue;
wherein the flexible wire is radiopaque and wherein the tip has a J-hook.

10. The device of claim 9, wherein the catheter further comprises a sheath configured to enclose the shunt device and the flexible wire prior to delivery of the shunt device, and wherein the flexible wire is received in an opening of the proximal arm of the shunt device.

11. The device of claim 9, wherein the flexible wire comprises a proximal end configured to be manipulated by a user to advance and retract the flexible wire, and wherein the flexible wire has sufficient rigidity to enable tactile detection, by a user, of deflection of the flexible wire.

12. The device of claim 9, wherein the flexible wire comprises a nitinol core.

13. The device of claim 9, wherein the flexible wire has an atraumatic tip for preventing puncture of the human tissue.

14. The device of claim 9, wherein the flexible wire is configured to be spaced apart from the distal arm when the shunt device is properly positioned having the distal arm on a first side of the tissue wall and the proximal arm on a second side of the tissue wall.

15. The device of claim 9, wherein the catheter further comprises a tube extending into the side opening, wherein the flexible wire is slidably received in the tube.

16. The device of claim 15, wherein the tube comprises a braided tube co-molded with the catheter, wherein the braided tube has an opening at a distal end configured to deliver a contrast agent, and wherein the opening is disposed adjacent to the central flow tube of the shunt device when the shunt device is seated.

17. A device comprising:
a catheter comprising a tube extendable outward therefrom, the tube having an opening at a distal end;
a shunt device disposed on the catheter, the shunt device comprising:
a central flow tube;
a first pair of arms connected to a first side of the central flow tube and configured to capture a tissue wall therebetween; and
a second pair of arms connected to a second side of the central flow tube and configured to capture a tissue wall therebetween, the second side opposite the first side;
a flexible wire slidably received in the tube and extendable through the opening of the tube, the flexible wire having a tip at a distal end for deflecting upon contact with human tissue.

18. The device of claim 17, wherein the flexible wire is radiopaque and comprises a proximal end configured to be manipulated by a user to advance and retract the flexible wire through the tube, and wherein the flexible wire has sufficient rigidity to enable tactile detection, by a user, of deflection of the flexible wire.

19. The device of claim 17, wherein the flexible wire is slidably received in an opening of an arm of the first pair of arms.

20. The device of claim 17, wherein the tube comprises a braided tube co-molded with the catheter.
